(19) United States
(12) Patent Application Publication
(10) Pub. No.: US 2005/0002962 A1
(43) Pub. Date: Jan. 6, 2005

(54) Immunostimulatory agents in botanicals

(76) Inventors: David Pasco, Oxford, MS (US); Nirmal D. Pugh, Oxford, MS (US); Ikhlas Khan, Oxford, MS (US); Rita Moraes, Oxford, MS (US)

Correspondence Address: Richard S. Myers, Jr.;Stites & Harbison PLLC, Suite 1800, 424 Church Street, Nashville, TN, 37219-2376, US

(21) Appl. No.: 10/826,559

(22) Filed: Apr. 16, 2004

Related U.S. Application Data
(60) Provisional application No. 60/463,169, filed on Apr. 16, 2003.
(60) Provisional application No. 60/538,676, filed on Jan. 23, 2004.

(51) Int. Cl. A61K 35/84; A61K 35/78

(52) U.S. Cl. 424/195.15; 424/729; 424/728; 424/726; 424/737; 424/756; 424/757; 424/764; 424/762; 424/771

(57) ABSTRACT

A melanin preparation as an immunostimulatory agent from at least one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom). Also disclosed is methods of treating a subject requiring immune mediation comprising administering to said subject a therapeutically effective amount of a melanin preparation from any one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom), Boletus edulis (porcini mushroom).

### PRIORITY

This application claims priority to U.S. patent application Ser. Nos. 60/463,169, filed Apr. 16, 2003, and 60/538,676 filed Jan. 23, 2004. The contents of both of these application are incorporated herein by reference in their entirety.

### GOVERNMENT SUPPORT

This invention was made with support from Grant Number AT001207 from the National Institutes of Health National Center for Complementary and Alternative Medicine and Specific Cooperative Agreement No. 58-6408-2-0009 from the United States Department of Agriculture. The United States Government has rights to this invention.

### FIELD OF THE INVENTION

The present invention relates to the field of immunostimulatory agents, and more particularly to melanin preparations isolated fromEchinacea species and other botanicals that are activators of immune cells. Further, the melanin preparations of the present invention can be used as immune system modulators, as pharmaceutical agents, or as dietary supplements. Quantitation of melanin content and activity within botanicals could form the basis for standardized, consistent products for the consumer market and for use in clinical trials. The present invention is also directed to procedures for enhancing melanin activity within botanicals and for isolating immuno-active melanin material and melanin preparations.

From a pharmaceutical perspective, most known immunostimulants are polysaccharides, glycoproteins, lipopolysaccharides, microbial products, and biologicals (e.g., interferons, TNF, CSF, and interleukins). The potential pharmaceutical use of Echinacea and other botanical melanin preparations described in the present invention represent a new class of immunostimulants.

### BACKGROUND OF THE INVENTION

Many of the most widely used dietary supplements in the United States and Europe are promoted for their immune enhancing properties. For many of these supplements, including botanicals, the compounds responsible for immune promoting effects have not been identified. An example of this relates to products containing Echinacea species, in that little evidence exists supporting the clinical relevance of the compounds that are used to standardize these products. A further complication is that Echinacea products can vary with respect to the species used (E. purpurea, E. angustifolia, E. pallida), the plant part (roots, aerial and whole), and the formulation (encapsulated powder, expressed juice and alcoholic extracted tinctures). Since it has been shown that different Echinacea preparations can have distinctly different chemistries, it would be expected that different pharmacological effects would result from use of different product types. This level of formulation complexity and therefore chemistry contributes to the problem in interpreting clinical trials performed to date on these different preparations. It also underscores the importance of identifying the clinically relevant compounds within the different preparations so that standardized consistent products could be produced for the consumer market and for use in clinical trials.

The present inventors have discovered melanins within Echinacea species that is present at high levels and is a potent activator of monocytes. Without being bound by theory, this material is likely responsible for the bulk of this type of biological activity within this herb when compared to the levels and activities of compounds presently viewed as being responsible for these immunostimulatory properties. In addition, the present inventors have discovered that there is a wide range in immunostimulatory activity among melanins isolated from the most commonly used botanicals and that elicitors that are presently used agriculturally greatly enhance the immunostimulatory activity of these melanins.

As an example, the following paragraphs will summarize current pertinent research on the role of different chemical constituents in the pharmacological action ofEchinacea on immune function, and why this newly discovered material has evaded detection until now.

A. Previous Studies on Immunomodulatory Echinacea Components

Several different candidates have been identified from Echinacea that may contribute to its immunomodulatory effects, including polysaccharides of various sizes, caffeic acid derivatives and alkamides. The most studied and best corroborated compounds are the polysaccharides, with supporting evidence coming from studies conducted both in vitro and in vivo (reviewed in Emmendorffer et al, 1999). Research on the alkamides also indicate a major role for these compounds. Much less evidence exists for the immunostimulatory actions of the caffeic acid derivatives. In all likelihood the combined actions of these and other unknown agents on multiple targets contribute to the overall therapeutic activity of Echinacea products.

1. Polysaccharides

The earliest studies on Echinacea polysaccharides were conducted using a preparation of varying degrees of purity that was isolated from aerial parts and roots of E. purpurea (Wagner and Proksch, 1981). Further purification yielded a protein-free preparation called EPS (Stimpel et al, 1984) and two polysaccharides; PSI, 4-O-methylglucuronoarabinoxylan (35,000 Daltons) and PSII, a 50,000 Dalton acidic arabinorhamnogalactan (Proksch and Wagner, 1987). These polysaccharides did not influence T and B cell proliferation or cytokine production but instead affected the phagocytosis, chemotaxis, and production of cytokines observed in granulocytes and macrophages (Stimpel et al, 1984; Wagner et al, 1985). These polysaccharides also enhanced the cytotoxic action of macrophages toward tumor P815 cells (Stimpel et al, 1984). Later work repeated and extended earlier studies by using an arabinogalactan isolated from E. purpurea grown in tissue culture (Wagner et al, 1988). This polysaccharide enhanced intracellular killing of Leishmania enriettii (Luettig et al, 1989) and Candida albicans (Lohmann-Matthes and Wagner, 1989).

Although the evidence supporting the in vitro effect on immune cells of Echinacea polysaccharides discussed above appears convincing, the concentrations required to obtain these effects were extremely high. For example, in studies using EPS (Stimpel et al, 1984) concentrations of 1,000 μg/ml were required to enhance macrophage cytotoxicity. In addition, this high concentration of EPS was required to enhance macrophage IL-1 production to levels 50% of those achieved using maximal concentrations of Salmonella lipopolysaccharide (LPS). Similarly, even high concentrations (250 μg/ml) of the purified arabinogalactan isolated from cultures of suspension cells of E. purpurea (Wagner et al, 1988) were required to enhance macrophage production of cytokines to levels equal to (interferon-β) or 20% (IL-1) of those achieved with maximal concentrations of E. coli lipopolysaccharide (10 μg/ml). Considering that crude preparations of these polysaccharides comprise less than 1% of the dry weight of Echinacea material (Stimpel et al, 1984) it suggests that more in vivo studies are needed to assess the contribution that Echinacea polysaccharides make to the overall immunostimulatory action of this botanical. To answer this question, levels of polysaccharides should be administered orally to animals at levels reflecting their content in ground Echinacea plant material or within Echinacea extracts. Most of the studies listed below use Echinacea polysaccharides injected i.v. into mice and are most likely not relevant to their action when taken orally.

Animal studies using i.v. EPS demonstrated enhanced phagocytosis (Wagner et al, 1985) and the arabinogalactan (administered i.v.) enhanced colony stimulating factor production in mice (Lohmann-Matthes and Wagner, 1989). Arabinogalactan injected i.v. into mice exhibited enhanced resistance against systemic infections with Listeria monocytogenes and Candida albicans in both normal (Roesler et al, 1991) and immunocompromised (Steinmuller et al, 1993) animals. More recently, oral administration of a polysaccharide fraction from E. purpurea aerial parts had no effect on lung macrophage function in normal rats (Goel et al, 2002). A pilot study using polysaccharides purified from E. purpurea tissue culture that were injected i.v. into patients undergoing chemotherapy for gastric cancer showed a lessening of leucopenia (Melchart et al, 2002).

2. Caffeic Acid Derivatives

(a) Cichoric acid

Cichoric acid is present in roots of E. purpurea (0.6%-2.1%) and aerial parts of E. purpurea, E. angustifolia and E. pallida at concentrations of 1.2-3.1% (Bauer et al, 1988c). In an in vitro granulocyte assay, cichoric acid concentrations between 10 and 100 ng/ml caused strong stimulation of phagocytosis and in mice it enhanced carbon clearance (Bauer et al, 1989a). Although cichoric acid may influence some aspects of immunity more studies are required to verify this.

(b) Echinacoside

The highest concentrations (0.3-1.7% dry weight) of echinacoside, a major polar constituent, occurs in the roots of E. angustifolia and E. pallida roots (Bauer and Remiger, 1989). Lower levels have also been reported in flowers of E. angustifolia (0.1-1%) and trace amounts in E. pallida flowers (Bauer et al, 1988c). In contrast, echinacoside is not present in E. purpurea root or aerial parts (Bauer et al, 1988c; Pietta et al, 1998).

Evidence is lacking for a role of this compound in immune stimulation as it was inactive in the carbon clearance test (Bauer et al, 1989a). Bauer also states in a recent review that “. . . as far as we know, it [echinacoside] does not possess any immunomodulatory relevance” (Bauer, 1999).

3. Alkamides

One of the major lipophilic components of Echinacea is the alkamides and the aerial parts of all three species contain these compounds (Bauer et al, 1988c). Fifteen major alkamides were identified in roots of E. angustifolia (Bauer et al, 1989b) and 11 in E. purpurea roots (Bauer et al, 1988c). In contrast, the roots of E. pallida do not contain alkamides but have high levels of ketoalkenes and ketoalkynes (Bauer et al, 1988b).

The following studies were performed to determine if the phagocytic activity exhibited by extracts from Echinacea was predominantly due to polar or non-polar compounds. In these studies ethanolic extracts from all three species and from both roots and aerial parts were separated into a polar (water) and non-polar (chloroform) fraction. The fractions were tested for phagocytosis in the granulocyte smear test (in vitro) and carbon clearance (in vivo). In essentially every case the non-polar (chloroform) fractions were the most active (Bauer et al, 1988a; Bauer et al, 1989a). A further purified non-polar fraction enriched for alkamides (isolated from E. purpurea and E. angustifolia roots) enhanced phagocytosis in the carbon clearance test by 1.5 to 1.7 times (Bauer et al, 1989a). Since an unpurified alkamide fraction exhibited this immunostimulatory effect, one cannot conclude that the alkamides were responsible for the activity. In a recent review by the author of this study the following was stated: “Since the main constituent, dodecatetraenoic acid-isobutylamide, exhibits only weak activity, the most effective constituents remain to be found” (Bauer, 2000). In a recent study, a purified alkamide fraction was administered orally to rats and was found to enhance the phagocytic activity and phagocytic index of lung alveolar macrophages. In addition, alveolar macrophages collected from alkamide-treated rats produced more TNF-α and nitric oxide after stimulation with LPS in vitro (Goel et al, 2002).

It has also been shown that purified alkamides (isolated from E. angustifolia roots) inhibited activity of both cyclooxygenase I and II as well as 5-lipoxygenase (Muller-Jakic et al, 1994; Clifford et al, 2002). It has been widely observed that many agents that inhibit cyclooxygenase activity also inhibit monocyte activation (Housby et al, 1999). Further research will be required to determine the relevance of the anti-inflammatory activity of alkamides to the clinical efficacy of Echinacea.

B. Monocyte Test System

In light of the valuable role the monocyte/macrophage plays in host resistance and since many of the previously reported activities using Echinacea suggested that monocyte/macrophage activity was influenced, the present inventors chose this cell type to base an assay to aid in the identification of compounds within Echinacea that could modify immunity. For monocytes to play a major role in adaptive and innate immunity they must respond effectively to environmental agents by first becoming activated (Adams and Hamilton, 1992). A major mediator of this activation is the proinflammatory transcription factor NF-kappa B. In unstimulated monocytes/macrophages NF-kappa B exists as inactive heterodimers sequestered by inhibitory-kappa B (I-kappa B) proteins within the cytosol. Agents that cause I-kappa B proteins to dissociate and degrade allow for the translocation of NF-kappa B dimers to the nucleus where they can activate transcription of downstream genes (May and Ghosh, 1998). Target genes regulated by NF-kappa B include proinflammatory cytokines, chemokines, inflammatory enzymes, adhesion molecules and receptors (Baeuerle and Henkel, 1994). In the assay that the present inventors developed NF-kappa B activation serves as a sensitive and rapid readout of the degree of monocyte activation.

The present inventors have employed this NF-kappa B/luciferase reporter gene based assay to screen for immunomodulatory activities within extracts from commercially available herbal products, plants and marine organisms from the natural product repository at the National Center for Natural Products, The University of Mississippi, University, Miss. This assay uses one of the most widely studied human monocyte cell lines, THP-1. A DNA plasmid is introduced into these cells made up of a luciferase reporter gene and two copies of a binding site for the NF-kappa B transcription factor. In this system the degree of activation of NF-kappa B, as determined by light output mediated by luciferase enzyme expression, corresponds quite closely with the induction of cytokine mRNAs and therefore the state of activation. This assay is extremely sensitive in that there is a 300- to 400-fold difference in induction of luciferase activity between untreated THP-1 cells and those treated with maximally activating levels of LPS. It is also an ideal system for testing natural product extracts, since only a four hour exposure is required to detect maximal activation of NF-kappa B. This system has enabled the present inventors to make important contributions to the understanding of components within several botanicals and other natural products that can modify monocyte activation and therefore may impact other aspects of the non-specific immune system.

C. Melanins are Complex Pigment Polymers that occur Throughout Nature

Melanins are complex polymers, and like other biomacromolecules, can be divided into classes. Typically melanins have been classified according to origin of material, extractability, solvent solubility, and chemical structure of subunits. For example, in mammalian tissue the two main types of melanin are eumelanins (black colored material that is insoluble in most solvents) and pheomelanins (yellow or reddish brown, alkali-soluble pigments). Biosynthetically, these animal melanins are derived from tyrosine oxidation by tyrosinase (coupled with thiols such as glutathione or cysteine for pheomelanins). In microorganisms, such as fungi, most melanins are derived from 1,8-dihydroxynaphthalene and contribute to virulence and modification of host immune responses (Gomez and Nosanchuk, 2003). Although a few reports have been published on plant melanins, definitive structural data is often lacking. One type of melanin isolated from plants is called “allomelanin” which is defined as a nitrogen-free polymer composed of phenol linked to proteins (Kamei et al, 1997). Some of the biological effects attributed to melanins include direct acting antiviral activity (Montefiori and Zhou, 1991) and the more commonly known photoprotective/redox properties (Riley, 1997). The inventors have found only one report of a “melanin-like” material exhibiting immunostimulatory activity. However, this material could not be conclusively identified as a melanin since no composition data was provided to indicate what the structural units were which composed this substance. This material was isolated from black tea leaves and when orally administered to mice, enhanced the antibody response of spleen cells to sheep red blood cells in as little as two days (Sava et al, 2001). The present inventors have found that green tea contains melanin that has unexpectedly superior activity compared to black tea melanin isolated by the inventors. This suggests that the material described by Sava et al. is not a melanin product similar in nature to those of the present invention.

The insolubility of melanins in common solvents has been a major obstacle in both initial extraction as well as purification schemes. Two general isolation approaches have been developed. The first approach isolates melanin by removal of all other substances from the initial material. This elimination process typically involves harsh chemical treatments with strong acids and base. The major problems with this approach is that the melanins remain contaminated with other classes of compounds and the harsh isolation conditions leads to the destruction of native melanin structure. The second approach extracts alkali soluble melanins with either strong base at high temperatures (for example 0.5 to 3 M sodium hydroxide) or weak base (2% ammonium hydroxide) at room temperature. The immune stimulatory properties of some melanins may have been missed due to harsh treatment with base since the present inventors have found that treatment of melanin from Echinacea and other botanicals with 0.5 M sodium hydroxide completely destroys its ability to activate monocytes.

Structural characterization of melanin polymers has been notoriously difficult due to their general insolubility in most solvents. Although aggressive chemical degradation methods have been proposed, these procedures have been severely limited by poor reproducibility, possible artifacts and low yield of degradation products (Dzierzega-Lecznar et al, 2002; Vas et al, 1993). Furthermore, the use of NMR to gain structural information on intact melanin polymers has been limited due to the high molecular weight and poor solubility of this material. Although solid-state ¹³C and ¹⁵N NMR have been used to decipher some information on functional groups (Knicker et al, 1995), structural assignment of monomer units and linkages has not been possible. One alternative to chemical degradation is pyrolysis which has been successfully used in melanin identification and to analyze structural differences between melanins (Dzierzega-Lecznar et al, 2002).

The inventors have found that Echinacea melanin can be extracted using weak base and under these conditions it retains its ability to activate monocytes. The sensitivity to strong base in addition to melanins limited solubility in commonly used solvents may explain why previous investigators did not detect this potent immunostimulatory material in Echinacea and other botanicals. The present inventors have developed an efficient and quantitative isolation procedure based on initial extraction with aqueous phenol that results in melanin preparations from plant material of high purity while maintaining its immunological activity.

### SUMMARY AND OBJECTS OF THE INVENTION

The present inventors have discovered melanins within Echinacea species and other botanicals that are present in high levels (up to about 10%) and are potent activators of monocytes. Without being bound by theory, this material are likely responsible for the bulk of this type of biological activity within some of these herbs when compared to the levels and activities of compounds presently viewed as being responsible for these immunostimulatory properties. Although melanins have been shown to be present in some plants, not all of the melanins that the present inventors have isolated will activate monocytes. The main reason why this material has not been previously identified as an important immunostimulatory component of botanicals is its lack of solubility in solvents normally used to extract plant material and its sensitivity to strong base.

Therefore, one object of the present invention is to provide a melanin preparation as an immunostimulatory agent from at least one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

Another object of the present invention is to provide an immunostimulatory composition that consists essentially of an immunostimulating effective amount of a melanin preparation from at least one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

Another object of the present invention is to provide an immunostimulatory composition that comprises an immunostimulating effective amount of a melanin preparation from at least one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

The preparations, agents, compositions of the present invention may be combined with a pharmaceutical carrier.

Other objects of the present invention include preparations that comprise the above-described melanin preparations.

One embodiment of the present invention is to provide a method of treating a subject requiring immune mediation comprising administering to said subject an immunostimulating effective amount of a melanin preparation, including those prepared or extracted from at least one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

Another embodiment of the present invention is to provide an extract having the ability to activate immune cells (such as monocytes) and consisting primarily of melanin from at least one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

Another embodiment of the present invention is to provide a standard for measuring and/or standardizing an effective amount of an immunostimulating agent in at least one of Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom). This method may comprise the following steps:
a. extracting a plant material with aqueous phenol;
b. collection of a precipitate, which comprises melanins;
c. removal of contaminants by at least one solvent wash;
d. removal of contaminants by partitioning, such as by phenol:water partitioning;
e. collection of melanin precipitation, including melanins from phenol layer;
f. testing the precipitate for activation of immune cells.

Another object of the present invention is to provide a method of preparing melanin or an extract enriched for melanin from at least one of Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom) using aqueous phenol (or chemically similar solvent), weak basic solvent (such as ammonium hydroxide), water, or aqueous alcohol (wherein the alcohol concentration ranges from 0-100%) extraction to be used for consumption by a subject.

Another object of the present invention is to provide a method for enhancing the immunostimulatory activity of melanin within the botanicals disclosed herein by treatment of said botanical with elicitors commonly used for agricultural purposes.

These and other objects will be apparent from this disclosure.

### BRIEF DESCRIPTION OF THE FIGURES

FIG. 1 is an infrared spectra of Echinacea melanin. In connection with this Figure, melanin was extracted from cloned E. angustifolia using the phenol procedure. KBr sample was prepared with 1 mg of melanin plus 100 mg of spectrometric grade KBr. Absorbances at 2360.2 cm⁻¹ and 2341.0 cm⁻¹ are due to carbon dioxide.

FIG. 2 shows a total ion chromatogram of melanin pyrolysis products. Here, melanin was extracted using the phenol procedure from A. Echinacea angustifolia cloned in vitro propagated plants or B. commercially obtained alfalfa sprouts. Samples were analyzed by Pyrolysis-GC-MS using a CDS Pyroprobe®D 2000 at 700° C. for 10 seconds with a temperature rise of 10° C./millisecond. Compound identification was accomplished by comparison with mass spectra from the Wiley library. Peaks correspond to the following thermal decomposition products: toluene (1), ethylbenzene (2), 3-methylpyrrole (3), styrene (4), phenol (5), 4-methylphenol (6), benzene acetonitrile (7), benzene propanenitrile (8), indole (9), 7-methylindole (10).

FIG. 3 shows a response of THP-1 monocytes to Echinacea melanin. A. Melanin was extracted from cloned E. angustifolia using the phenol procedure. Twenty-four hours following transfection with the NF-kappa B luciferase reporter plasmid, cells were treated with the indicated agents for 4 hours. Luciferase activity was determined and is reported as percent of maximal light output from LPS-treated cells. Values represent the average of duplicate determinations. B. THP-1 cells were incubated for 2 hours with Echinacea melanin (ECH-20 μg/ml) or LPS (10 μg/ml). Cells were harvested and total RNA was isolated and processed by RT-PCR using primers specific for the mRNAs indicated in the figure.

FIG. 4 demonstrates the effect of TLR blocking antibodies on THP-1 activation. THP-1 cells were treated with antibodies to CD14 (MY4), TLR2, TLR4, or control IgG fractions for these antibodies (MsIgG2b, IgG2a) for 30 min. prior to addition of specified agent. Four hours later cells were harvested for luciferase assay. The results are the average of two experiments with each sample performed in duplicate. A. Data for Echinacea melanin, Ultra Pure LPS and microalgae polysaccharide. B. Data for Alfalfa and American Ginseng melanin.

FIG. 5 shows monocyte activity for the water soluble and phenol soluble components in each mushroom. Two crude extracts were prepared from each mushroom: extraction of freeze-dried material twice with hot water at 70° C. and extraction of freeze-dried material twice with 90% aqueous phenol at 70° C. Hot water extracts were solvent partitioned once against phenol and phenol extracts were solvent partitioned once against water. Water layer and phenol layer fractions were evaluated in the monocyte test system at concentrations of 2 and 20 μg/ml.

FIG. 6 was shows total ion chromatogram of Agaricus bisporus mushroom melanin pyrolysis products. Melanin extracted from Agaricus bisporus (the common commercial) mushroom using the phenol procedure. Sample was analyzed by Pyrolysis-GC-MS using a CDS Pyroprobe® 2000 at 700° C. for 10 seconds with a temperature rise of 10° C./millisecond. Compound identification was accomplished by comparison with mass spectra from the Wiley library. Peaks correspond to the following thermal decomposition products: toluene (1), ethylbenzene (2), 3-methylpyrrole (3), styrene (4), phenol (5), 4-methylphenol (6), benzene acetonitrile (7), benzene propanenitrile (8), indole (9), 7-methylindole (10).

FIG. 7 shows time course of enhancement of melanin activity by treatment with chitin. Alfalfa sprouts were treated with chitin (50 mg/ml) for 12 hours. At the specified time the sprouts were freeze dried and melanin was extracted using the phenol procedure. The data represent the average of triplicate samples from one experiment that was repeated two times. Harvest time of 0 hours indicates the termination of the 12 hour chitin treatment period. The melanin was used at a concentration of 100 ng/ml in the monocyte activation assay.

FIG. 8 shows the effect of American Ginseng melanin on immune parameters. Five month old, male, C3H/He mice (4 per treatment group) were treated for 4 days with either 25 mg ginseng melanin/day (mixed with chemically defined chow AIN-76A) or treated with AIN-76A chow alone. A. Peyer's patch cells isolated from these mice were cultured for 3 days and the culture medium analyzed for IgA content by ELISA. B. Spleen cells isolated from these mice were cultured for 3 days and the culture medium analyzed for interferon gamma by ELISA. Values are averages ± standard deviations and statistical analysis was by a Student's t-test.

### DETAILED DESCRIPTION OF THE INVENTION

A. Discovery of a previously Unidentified Material within Echinacea that Activates Monocytes

During the initial studies with Echinacea material from various sources, the present inventors very rarely detected any activation in the monocyte test system no mater what type of solvent was used to extract the plant material. Initially, the present inventors thought that inhibitory (anti-inflammatory) compounds present might be masking the activating compounds within these extracts. However, in some experiments where stimulatory activity was detected upon initial testing of an extract, this activity would not be consistently seen upon retest of the extract. It became apparent that the reason for this inconsistency was a solubility problem since sonication of the previously tested extract would render the extract active. Without being bound by theory, this observation prompted the present inventors to hypothesize that the inability to detect monocyte-activating properties in these extracts might stem from the inability to solubilize and therefore extract the active material.

The present inventors tested this hypothesis in a very unorthodox manner. The present inventors suspended finely ground root material from E. purpurea in 95% ethanol, and subsequent to the settling out of the bulk of the material, they added the remaining suspension to the monocyte test system. Upon microscopic examination this suspension contained a fine mist of microscopic particles. The present inventors thought that since most factors that activate monocytes do so through interaction with cell surface receptors, that it may be possible that the activating substance may be “bio-available” on the surface of these particles. When this suspension was added to the monocytes it resulted in substantial activation (30% of maximal activation). If the particulate suspension was filtered through a 0.22 μm filter or centrifuged at 16,000× g for 5 minutes prior to addition to the monocytes, no activation was observed. This indicated that the activating substance did not dissolve in the 95% ethanol and remained with the particulate material. Because it was possible that the particulate nature of the material was responsible for activation and not the composition of the particles, other plant material was tested in an identical manner. Material (both aerial and root) from 10 plants did not exhibit any activation despite a similar or greater concentration of particulate matter suggesting that something about the composition of the Echinacea particles was unique.

An extensive series of solvents were tested for efficient extraction of this activity and included the following: hexane, ethyl acetate, chloroform, 95% ethanol, methanol, isopropanol, dimethyformamide, and dichloromethane. All of these solvents failed to efficiently extract the activity. Partial success was obtained with water, and extraction was further enhanced with 50% ethanol or pyridine/water (5:2) at 70° C. These conditions were extremely inefficient however, since each of 5 sequential extractions yielded similar yields and activities (˜25% activation at 50 μg/ml). Since these extraction conditions suggested that the active contained both polar and nonpolar properties, it was hypothesized that the compound might be amphipathic. Imunostimulants that exhibit this property include lipopolysaccharides and sphingolipids.

The classical method for extracting lipopolysaccharides involves extraction using 50-90% phenol at 65-70° C. This is followed by either precipitation from phenol or partition into the water layer depending on the lipid content of the compound (Galanos et al, 1969; and Westphal and Jann, 1965). This approach proved very effective for the isolation of this active material because the marc material was essentially inactive when tested in suspension in the monocyte test system. Cloned E. angustifolia propagated under aseptic conditions was used for these experiments to exclude the possibility of contamination with microbial derived lipopolysaccharide-like material. The material was extracted 4 times with 90% aqueous phenol at 70° C. for 30 minutes each and active material precipitated with 6 volumes of ether/acetone (1:5). The bulk of the precipitable material was obtained in the first two extractions. The precipitate was washed several times with ethyl acetate and isopropanol, redissolved in 90% phenol, and then partitioned against water. Essentially all of the activity remained with the phenol layer and was recovered by precipitation. This material constituted 4 to 5% of the dry plant material and exhibited an activity of 50% activation at between 1 and 3 μg/ml. This material is extremely soluble in 90% aqueous phenol at concentrations of 100 mg/ml. Solvents with properties similar to phenol (benzyl alcohol, 2-phenylethanol and 1 -octanol) were less effective although beenzyl alcohol showed some ability to solubilize this material. The following solvents were all ineffective at dissolving this material: DMSO, 1,4-dioxane, tetrahydrofuran, acetonitrile, ethylene glycol, and propylene glycol.

Analysis of this material by The University of Georgia Complex Carbohydrate Research Center revealed that it contained less than 1% carbohydrate and therefore indicated that it was not a lipopolysaccharide or polysaccharide. Treatment of the material at 37° C. for one hour with DNase I, RNase A, Proteinase K, Trypsin, or α-Chymotrypsin did not decrease the activity of this material in the monocyte assay indicating that the activity was not due to a nucleic acid or protein. More extensive treatment of this material by heating at 98° C. for 2 hours or incubation with RNase A, Proteinase K, Pronase E, or bacterial Proteinase (Nagarse) at 1.0 mg/ml for 24 hours also did not result in loss of activity.

The following physical data indicated that the material is a melanin: amorphous dark color pigment (reddish brown and similar to pheomelanin), general insolubility in most solvents, bleaching by oxidizing agents (H₂O₂), and pheomelanin-like solubility in alkali and phenol (Krysciak, 1985). Elemental analysis indicated about 50% carbon, about 13% nitrogen, about 7% hydrogen, about 0.8% sulfur and about 0.08% phosphorus.

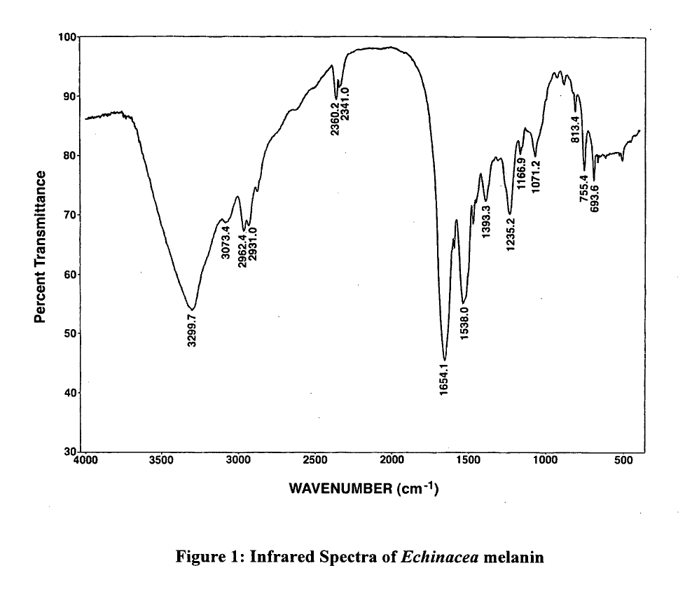

The IR spectrum for Echinacea melanin (FIG. 1) displays the major structural characteristics typical of other melanins: carbonyl groups and/or aromatic ring systems are indicated by strong absorbances at 1654.1 cm⁻¹ and 1538.0 cm⁻¹; hydroxyl (and possibly amino) groups by the broad band at 3299.7 cm⁻¹. Analysis of IR spectra for melanins isolated from other cloned Echinacea species were essentially identical to the characteristics displayed in FIG. 1. Therefore, since the monocyte stimulatory activity of these melanins vary considerably, the structural information provided by IR cannot be used to correlate with biological activity.

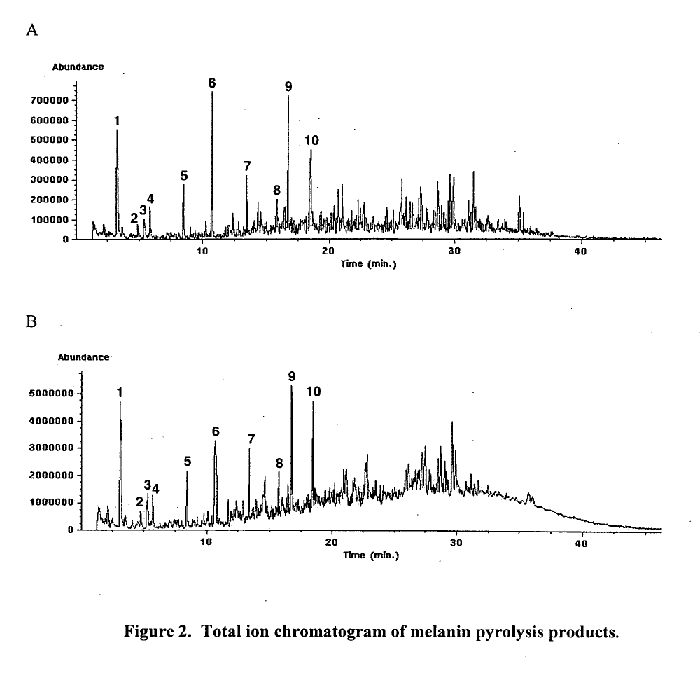

Structural analysis of Echinacea melanin using filament pyrolysis-GC-MS was based on established protocols (Vas et al, 1993; Zecca et al, 1992). Samples (0.1 mg) were analyzed by Pyrolysis-GC-MS using a CDS Pyroprobe® 2000 at 700° C. for 10 seconds with a temperature rise of 10° C./millisecond. Pyrolytic products were separated using a Hewlett Packard 5890 gas chromatograph using a HP-35 column (30 m×0.25 mm ID, film thickness 0.25 μm). The GC temperature conditions were as follows: initial temperature of 50° C. for 2 minutes; increased to 290° C. at a rate of 7° C./minute; and, the final temperature was held for 10 minutes. The gas chromatograph was coupled to a Hewlett Packard 5970B Quadrupole mass spectrometer operating under electron impact conditions (ionization energy of 70 eV). All mass spectra were recorded in the mass range between 50 and 650 AMU. Identification of pyrolytic products was accomplished by comparison with mass spectra from the Wiley library database. The total ion chromatogram of the pyrolysis products of Echinacea melanin is displayed in FIG. 2A.

The high temperature pyrolysis conditions used above are necessary for melanin thermal degradation since melanin is composed of covalently linked indoles. However, some proteins contain aromatic amino acids (tryptophan, tyrosine and phenylalanine) and these could give rise to some of the thermal degradation products identified above. For example, consistent with its substantially lower content of aromatics, pyrolysis of bovine serum albumin (same amount as used for melanin analysis) produced approximately 10 times less of the thermal degradation products seen with melanin preparations. Since melanin preparations isolated using the phenol extraction procedure are contaminated with varying amounts of protein (as determined by amino acid analysis of acid hydrolyzed material) it is possible that proteins are contributing to the pyrolysis signatures reported above.

The most common method for amino acid analysis of proteins is acid hydrolysis (Copeland, 1994). Typical conditions employed for hydrolysis involve incubating protein material for 20 hours at 115° C. in 6M HCl containing 0.05% mercaptoethanol and 0.02% phenol. A variety of separation and detection methods can be used to measure the amino acids resulting from protein hydrolysis. The total amount of amino acids provides a chemical method of determining the protein content.

However, as the inventors have described earlier, these proteins do not contribute to the monocyte activation properties of these preparations since extensive treatment with several types of proteases did not influence melanin activity. The inventors have identified conditions that can be employed to obtain protein-free melanin material. This protein-free melanin material can be collected during the water/phenol partitioning steps of the isolation procedure. During the successive partitions, due to reduction in phenol amounts and equilibration with water, phenol insoluble melanin precipitates out of solution. This phenol-insoluble melanin can be separated from material remaining in solution by centrifugation. Analysis of the phenol-insoluble melanin revealed that it was protein-free and gave an essentially identical pyrolysis thermal degradation profile to protein containing preparations (see FIG. 2B). In addition, phenol-insoluble melanin retained activity in the monocyte assay (data not shown). Other methods, as known in the art, such as degredation with proteases, may be used to remove proteins.

B. Procedures for Isolating Melanin with Consistent Activity and Yield from Echinacea and Other Plants

The procedure for isolating lipopolysaccharide material described above was adapted for the isolation of melanin from plant material. It was found that the procedure yields material of consistent activity and yield. Plant material is extracted with 90% aqueous phenol (1 g/22 ml) for 30 minutes at 70° C. and for the second extraction 16 ml is used, active material is precipitated with 6 volumes of ether/acetone (1:5). The precipitate is washed three times with ethyl acetate and twice with isopropanol. The pellet is redissolved in 90% phenol at 70° C. and undissolved material is removed by centrifugation at 3,000 rpm for 15 minutes. The phenol layer is then partitioned against equal volumes of water. The water partition is conducted at 70° C. and repeated until the top water layer is clear. The melanin material is precipitated from the phenol layer and interface as described above and washed extensively with isopropanol and dried under vacuum. Since this procedure appears to be very consistent and extraction complete, it would appropriately serve as part of a procedure for standardization to this immunostimulatory component.

A procedure used for isolation of plant melanins involves extraction with weak base, such as 2% ammonium hydroxide (Hung et al, 2002). This approach was capable of extracting active melanins from Echinacea and other botanical material but it was not as effective as the phenol procedure since the marc material contained significant phenol-extractable melanin. However, this approach would be sufficient for extracting this material for use for human consumption. Such an extraction procedure could be used to obtain active Echinacea or other botanical melanin that could then be used as is, used in combination with ground plant material to enhance its immunostimulatory activity or to “standardize” material so it contains a certain melanin level.

C. Variations in Content and Monocyte Stimulatory Activity of Melanins in different Echinacea Plants and Plant Parts

Table 1 demonstrates that the content and activity of melanins extracted from various plant parts and sources can vary substantially. In general, with cultivated Echinacea plants, the roots of E. pallida and E. purpurea contained melanin with substantially more activity than the aerial part while the leaves had higher activity than the roots in E. angustifolia. Leaves and cones have the highest content with stems and roots containing approximately half of this amount.

TABLE 1
Activity of melanin preparations extracted from the plant parts of Echinacea
species.

| PLANT | ROOTS | LEAVES | STEMS | CONES |
|---|---|---|---|---|
| cultivated | EC₅₀ (μg/ml) | | | |
| E. angustifolia | 50 (1.0) | 1-5 (1.3) | | |
| E. purpurea | 1-10 (0.7-1.9) | 500 (1.0-2.6) | 500 (1.4) | >1000 (2.6) |
| E. pallida | 5 (1.1) | 10 (2.8) | >1000 (1.8) | 100 (3.7) |
| Melanins were extracted and tested in the monocyte test system at concentrations of 1, 10 and 100 μg/ml. Values represent EC₅₀ (μg/ml) and are the concentration at which activation equaled 50% of that achieved with maximally activating concentration of LPS (10 μg/ml). Values in parentheses represent percent yield. For melanin preparations that exhibited less than 20% activation when run at 100 μg/ml, an EC₅₀ value of >1000 μg/ml is assigned since a | | | | |
| # doubling of percent activation requires an order of magnitude increase in melanin concentration in this assay system. | | | | |

D. Substantial Variations in Echinacea Melanin Immunostimulatory Activity among Clones from Three Echinacea Species Cultured under Aseptic Conditions

In addition to providing plant material free from possible microbial contamination, the use of cultured clones of the three commercially relevant Echinacea species also provides an unlimited source of genetically identical plants propagated under identical environmental conditions. If variations in the activity or content of melanins are detected among these clones this would suggest that genotypic differences were responsible for these variations. Table 2 presents data on these clones and shows that substantial differences were observed in the activity of the extracted melanin. Although some clones contained half as much melanin than the average, greater differences existed in the activity of this material. For E. angustifolia clones, the melanin from the leaves was substantially less active than the melanin extracted from the callus/stem.

TABLE 2
Content and activity of melanin preparations from in vitro propagated, Echinacea
species.

| Clone | EC₅₀ value (μg/ml) | | Content (% dry wt) | | Clone | EC₅₀ value (μg/ml) | | Content (% dry wt) | |
|---|---|---|---|---|---|---|---|---|---|
| No. | Callus/stem | Leaves | Callus/stem | Leaves | No. | Callus/stem | Leaves | Callus/stem | Leaves |
| EA16 | 1.6 | 15 | 8.3 | 6.4 | EPP26 | 9.3 | NT | 6.6 | NT |
| EA15 | 2.4 | 70 | 6.9 | 7.1 | EPP3 | 20 | NT | 6.8 | NT |
| EA17 | 2.7 | 150 | 6.2 | 6.4 | EPP1 | 100 | NT | 11.3 | NT |
| EA10 | 3.0 | 110 | 4.3 | 6.8 | EPP2 | >1000 | NT | 7.0 | NT |
| EA50 | 4.2 | 35 | 5.2 | 6.3 | EP9 | 2.5 | NT | 5.5 | NT |
| EA51 | 4.3 | 125 | 6.9 | 4.7 | EP12 | 15 | NT | 10.8 | NT |
| EA13 | 5.1 | 27 | 7.3 | 6.7 | EP5 | 100 | NT | 7.4 | NT |
| EA12 | 8.6 | 100 | 5.9 | 6.3 | | | | | |
| EA19 | >100 | >200 | 3.9 | 3.7 | | | | | |
| EA21 | >100 | >200 | 3.0 | 3.2 | | | | | |

Melanins were extracted with the phenol procedure and evaluated in the monocyte test.
EC₅₀ values represent the concentration at which activation equaled 50% of that achieved with maximally activating concentrations of LPS (10 μg/ml).
EA = E. angustifolia,
EPP = E. purpurea,
EP = E. pallida,
NT—not tested.

E. Relative Activity of Echinacea Immunostimulatory Polysaccharides and Melanins

Although the preceding suggests that melanins within some Echinacea species represent a significant source of immunostimulatory activity a comparison must be made with polysaccharide preparations from these plants. Because these melanins may have a slight solubility in water, Echinacea material extracted with hot water may also contain a small amount of this material in addition to previously reported monocyte-activating polysaccharides (Proksch and Wagner, 1987). To get an approximation of the relative contribution of these two materials the following experiment was performed. Cloned, in vitro propagated E. angustifolia leaves (100 mg) were extracted twice with water at 70° C. for 30 minutes each. The extract was pooled and split equally, one half was precipitated by the addition of ethanol and the other half partitioned with phenol. The present inventors assume that both materials contribute to the activity in the precipitate while partitioning separates the activities, melanins in the phenol layer and polysaccharides in the water layer. All samples were run in the monocyte test system at 10, 100 and 200 μg/ml. The precipitate gave 20% activation while the phenol and water layers gave 14% and 5%, respectively when run at 10 μg/ml. Since very little of the activity in the precipitate appears to be due to polysaccharides it suggests that these compounds contribute little compared to the melanins. The following analysis illustrates this view: The yield of melanins from this source is approximately 4% and when run at a concentration of 10 μg/ml usually results in activation from 90 to 100%. This would mean that 100 mg of this plant material would yield 400 maximal activations (4%×100 mg /0.01 mg=400). In contrast, the yield of the water layer from the water extract was approximately 3% and the concentration required to give maximal activation is estimated to be approximately 10,000 μg/ml. Using the same calculation and assuming the yield of polysaccharide material to be equal to that of the melanins, this fraction contributes 10,000 times less activity than the melanin (4%×100 mg /10 mg=0.4 maximal activations).

F. Sensitivity of Echinacea Melanin to Alkaline Conditions

The standard procedure for solubilization and extraction of pheomelanins from various sources is with aqueous solutions containing sodium or potassium hydroxide at concentrations ranging from 0.5 to 3.0 M. Because of problems experienced with solubility of Echinacea melanins the present inventors tried dissolving melanin material with 0.5 M NaOH. This melanin had been isolated from cloned E. angustifolia leaves using the standard phenol procedure. The alkaline conditions completely dissolved this material but when tested in the monocyte test system it was completely inactive. This sensitivity to base in addition to melanins insolubility in commonly used solvents may also explain why previous investigators did not detect this activity. In the initial studies on the isolation and characterization of Echinacea polysaccharides aqueous extracts were prepared by an overnight incubation in 0.5 M NaOH at room temperature followed by ethanol precipitation (Proksch and Wagner, 1987). These conditions would probably have inactivated most of melanins in the plant material and could therefore explain why this extremely potent immunostimulatory component was missed.

G. Echinacea Melanin Activates Monocytes through the NF-kappa B Transcription Factor Pathway and causes the Accumulation of IL-1β mRNA

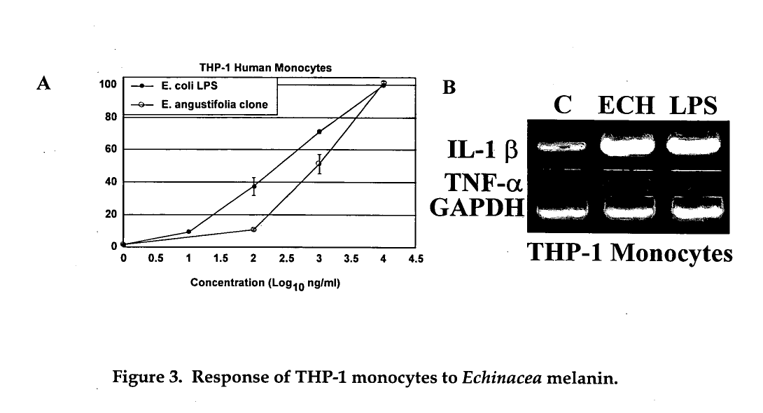

FIG. 3A compares the response to Echinacea melanin with that of the classical activator of monocytes E. coli LPS with respect to NF-kappa B activation in the human monocyte cell line THP-1. The EC₅₀ value for melanin was 1 μg/ml with maximal activation occurring at 10 μg/ml. Maximal activation with this melanin is equal to that of maximally activating concentrations of E. coli LPS (10 μg/ml). FIG. 3B confirms monocyte activation by Echinacea melanin in that this material substantially increased the expression of cytokine mRNAs characteristic of this state. Echinacea melanin induced IL-1μ mRNA expression to the same extent as maximally activating concentrations of LPS (10 μg/ml). These results lend further support for the view that Echinacea melanins represent a major immunostimulatory component of this plant.

H. Melanin Preparations from some, but not all Medicinal Plants, are Potent Activators of Monocytes in Comparison to Preparations from Commonly used Vegetables

Table 3 shows the activity in the monocyte assay of melanin preparations extracted from common herbs and vegetables. Several herbs (American ginseng root, black walnut hulls, green tea leaves, Parthenium root, Korean ginseng, alfalfa sprouts and ginger root) contained melanin that was 2-10 times more active than the melanins from the most active Echinacea material. The herbs tested in this study were selected because they are among the top sellers in the USA. In addition, over half of these herbs are traditionally used as immune stimulants and most of these contained melanin with activity similar to or more active than Echinacea melanin. None of the commonly used vegetables tested contained melanin with significant activity. The results are consistent with the view that melanins contribute to the immunostimulatory properties of other botanicals as well as Echinacea.

TABLE 3
Activity of melanin preparations extracted from selected herbs and common
vegetables.

| | EC₅₀ | Common | EC₅₀ |
|---|---|---|---|
| Common Herbs | (μg/ml) | Vegetables | (μg/ml) |
| American Ginseng root (Panax quinquefolius) | 0.1 | Swiss Chard stem | >1000 (19%) |
| Black Walnut hulls (Juglans nigra) | 0.1 | Red leaf lettuce | >1000 (18%) |
| Green Tea leaves (Camellia sinensis) | 0.2 | Carrot | >1000 (17%) |
| Parthenium integrifolium root | 0.3 | Iceberg lettuce | >1000 (10%) |
| Korean Ginseng root (Panax ginseng) | 0.4 | Green bean | >1000 (8%) |
| Alfalfa sprouts (Medicago sativa) | 0.4 | Spinach leaf | >1000 (8%) |
| Ginger root (Zingiber officinalis) | 0.5 | Celery stem | >1000 (4%) |
| Echinacea angustifolia leaf | 1.0 | Swiss Chard leaf | >1000 (3%) |
| Echinacea purpurea root | 1.0 | Broccoli floret | NA |
| Goldenseal root (Hydrastis canadensis) | 2.7 | Cabbage leaf | NA |
| Red Clover blossoms (Trifolium pretense) | 3.0 | Tomato | NA |
| Parthenium integrifolium leaf | 3.2 | Green bell pepper | NA |
| Dandelion shoot (Taraxacum officinale) | 3.2 | Green pea | NA |
| Black cohosh root (Actea recemosa) | 3.2 | White jasmine rice | NM |
| Licorice root (Glycyrrhiza glabra) | 3.5 | Red Potato | NM |
| Chamomile flower (Matricaria recuita) | 4.0 | Asparagus | NA |
| Milk Thistle seeds (Silybum marianum) | 4.4 | Butternut squash | NA |
| Echinacea pallida root | 5.0 | Yellow corn kernel | NA |
| Alfalfa herb (Medicago sativa) | 5.0 | | |
| Horsetail stems (Equisetum arvense) | 8.5 | | |
| Astragalus membranaceus root | 9.0 | | |
| Gotu Kola herb (Centella asiatica) | 15.0 | | |
| Feverfew herb (Tanacetum parthenium) | 25.0 | | |
| Valerian root (Valeriana officinalis) | 82.0 | | |
| Hawthorn fruits (Crataegus monogyna) | 100 | | |
| Black tea leaves (Camellia sinensis) | 500 | | |
| Rosemary leaves (Rosmarinus officinalis) | 1000 | | |
| Saw Palmetto berries (Serenoa repens) | 1000 | | |
| St. John's wort leaves (Hypericum perforatum) | >1000 (18%) | | |
| Garlic cloves (Allium sativum) | >1000 (14%) | | |
| Ginkgo biloba leaves | >1000 (12%) | | |
| Mahuang herb (Ephedra sinica) | >1000 (5%) | | |
| Pau D'arco inner bark (Tabebuia spp.) | >1000 (5%) | | |
| Agrimony herb | NA | | |
| Chickweed herb | NM | | |

Melanins were extracted using the phenol procedure and tested in the monocyte test system at concentrations ranging from 0.1 to 100 μg/ml.
EC₅₀ values represent the concentration at which activation equaled 50% of that achieved with maximally activating concentration of LPS (10 μg/ml). For melanin preparations that exhibited less than 20% activation when run at 100 μg/ml, percent activation is given in parenthesis. These preparations are assigned an EC₅₀ value of >1000 μg/ml since a doubling of percent activation requires an order of magnitude increase in melanin concentration in this assay system.
NA indicates not active at 100 μg/ml.
NM indicates no material was obtained. Percent recovery of melanin preparations for common herbs ranged from 0% to 9.3% and for vegetables 0% to 5%.

I. Toll-like Receptor 2 (TLR2) is involved in Monocyte Activation by Echinacea, Alfalfa and American Ginseng Melanins

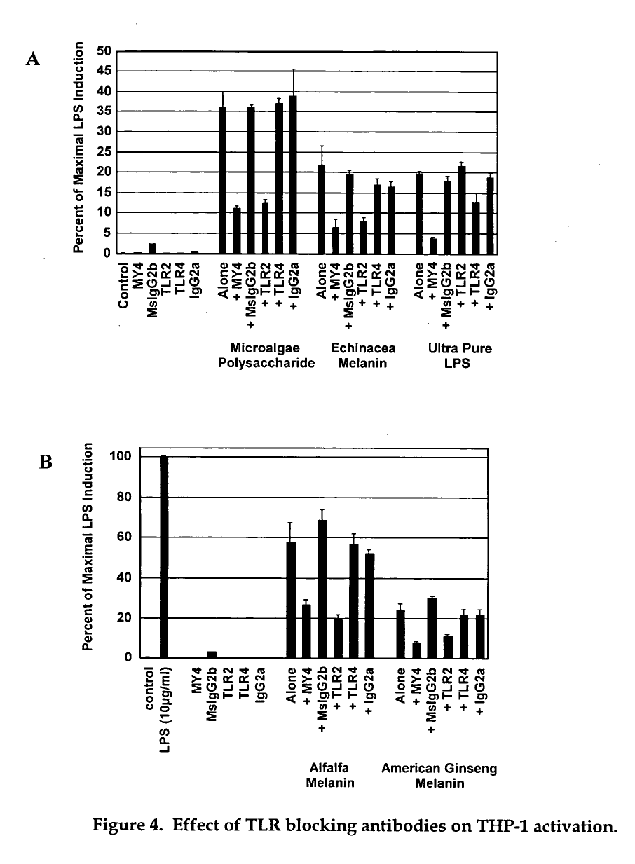

At present it is thought that many bacterial components such as lipopolysaccharides and lipoproteins are recognized by the innate immune system due to the binding of these agents to TLR2 and TLR4. The experiment presented in FIG. 4 suggests that TLR2 is involved in monocyte activation by melanins extracted from Echinacea, Alfalfa, and American Ginseng in that antibodies to this receptor suppress activation. Antibodies to CD14 also suppressed activation by these melanins consistent with its role in mediating the action of many TLR. TLR4 antibody was ineffective at suppressing melanin-dependent activation indicating the specificity of these antibodies. The control IgG fractions for these antibodies (MsIgG2b and IgG2a) also were not effective at suppressing activation. Activation by ultra pure Salmonella minnesota LPS (TLR4 ligand) was suppressed by TLR4 but not by TLR2 antibody (FIG. 4A). Activation by an extremely potent polysaccharide that the inventors have previously isolated from microalgae (Pugh et al, 2001) was also suppressed by TLR2 antibody (FIG. 4A). The modest suppression of activation seen with these blocking antibodies is typically observed by other investigators in these types of studies.

J. Melanin Preparations from Edible or Medicinal Mushrooms

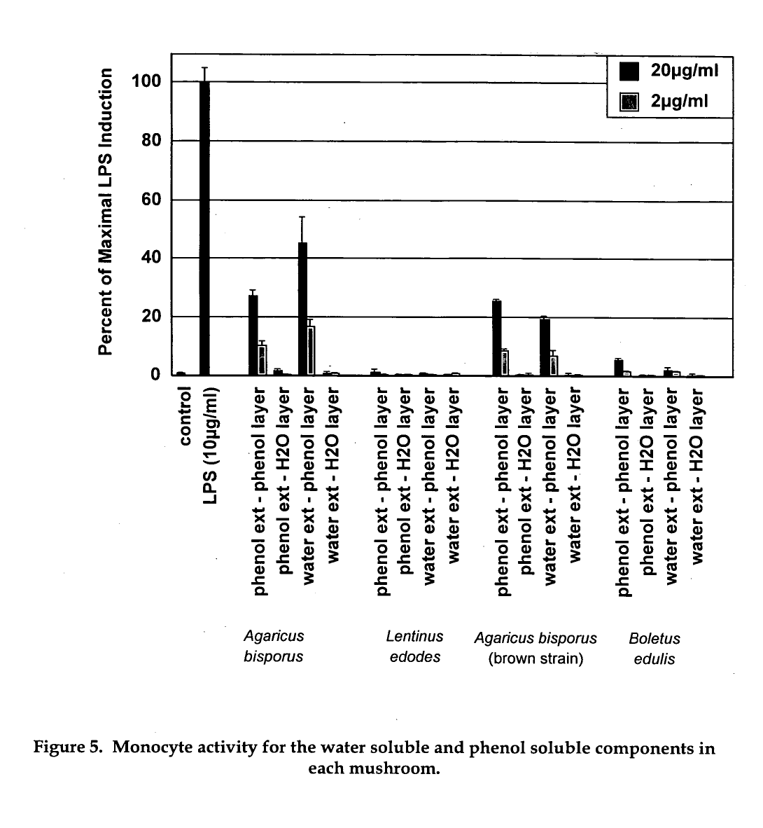

Mushrooms were produced by J-M Farms, Inc. (Miami, Okla.) and purchased from local grocery stores. Mushroom material was freeze-dried prior to use. To evaluate the relative contribution of water soluble components verses phenol soluble components to monocyte activation, two crude extracts were prepared from each mushroom. The first extract was prepared by extraction of mushroom material two times with hot water at 70° C. The hot water extract was then solvent partitioned one time against phenol. Components in the water layer were recovered by precipitation with 80% ethanol and components in the phenol layer were recovered by precipitation with six volumes of ether:acetone (1:5). The second extract was prepared by extraction of mushroom material two times with 90% aqueous phenol at 70° C. The crude phenol extract was then partitioned one time against water. Components in the water layer were recovered by reducing the sample to dryness and components in the phenol layer were recovered by precipitation with six volumes of ether:acetone (1:5). All water layer fractions (dissolved in water) and phenol layer fractions (resuspended in isopropanol) were evaluated in the monocyte test system at 2 and 20 μg/ml (FIG. 5). The phenol layer fractions contained essentially all of the immunostimulatory activity, while the water layer fractions were inactive. This suggests that very little of the activity in these mushroom is due to components in the water layer fractions (e.g. polysaccharides and proteins). This experiment also demonstrates that for the mushrooms tested, hot water extraction was as effective as 90% phenol for the extraction of melanin.

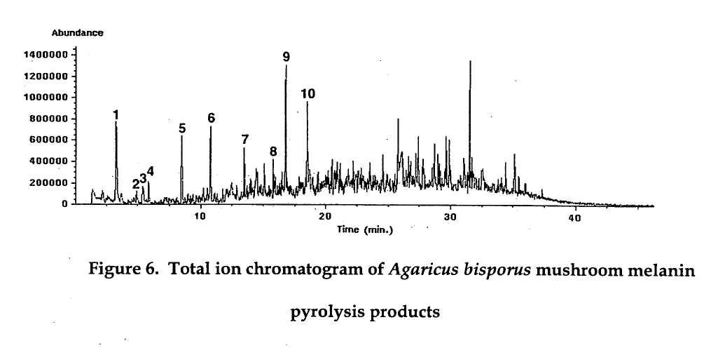

Based on the phenol extraction procedure for isolation of melanins, the phenol layer fraction from the phenol extract represent a crude melanin preparation. Analysis of this material for each mushroom using pyrolysis-GC-MS suggests a high content of melanin (a representative example from Agaricus bisporus is shown in FIG. 6). The EC₅₀ value for each mushroom melanin preparations is listed below. EC₅₀ values represent the concentration at which activation equaled 50% of that achieved with maximally activating concentration of LPS (10 μg/ml). For melanin preparations that exhibited less than 10% activation when run at 20 μg/ml, percent activation is given in parenthesis. These preparations are assigned an EC₅₀ value of >1000 μg/ml since a doubling of percent activation requires an order of magnitude increase in melanin concentration in this assay system. Percent recovery of melanin preparations for mushrooms ranged from 11.2% to 16.3%.

| | | |
|---|---|---|
| Estimated EC₅₀ value for Agaricus bisporus: | 30-100 | μg/ml |
| Estimated EC₅₀ value for Agaricus bisporus | 100-200 | μg/ml |
| (brown strain): | | |
| Estimated EC₅₀ value for Lentinus edodes: | >1000 | μg/ml (1%) |
| Estimated EC₅₀ value for Boletus edulis: | >1000 | μg/ml (6%) |

Extensive treatment of melanin isolated from Agaricus bisporus by heating at 98° C. for 2 hours or incubation with RNase A, Proteinase K, Pronase E, or bacterial Proteinase (Nagarse) at 1.0 mg/ml for 24 hours did not result in loss of activity. This indicates that this activity is not due to protein.

K. Differential Extractability/Solubility of Melanin Preparations from different Botanicals

Several experiments indicated that melanin preparations from different botanicals exhibit different solubilities and therefore solvent specific extractability. In section J, mushroom melanin was extracted quantitatively by both hot water and aqueous phenol. This was not consistent with the behavior observed with Echinacea melanin in that very little of the melanin material is extracted with hot water. In an additional experiment purified melanin material from alfalfa sprouts, American ginseng and cloned Echinacea angustifolia also exhibited different solubilities. For example, American ginseng melanin completely dissolved at a concentration of 10 mg/ml in weak base (0.035 or 0.25 % NH₄OH) while Alfalfa sprout and Echinacea melanin was approximately 10 times less soluble under these conditions. In water (pH 6), American ginseng melanin was slightly less soluble than under the weak base conditions while Alfalfa sprouts and Echinacea melanin exhibited almost no solubility. Adding increasing amounts of ethanol to the water (from 10 to 40% ethanol) decreased all three melanins solubilities. Although aqueous phenol (90%) appears to be the preferred or optimal extraction solvent for most botanicals, the use of weak base, water, aqueous ethanol or any combination of these solvents may be an effective alternative depending on the desired application. The concentration of weak base used to extract melanin is understood to be enough to effectively solubilize the melanin but not enough to cause inactivitation. Extraction of melanin with aqueous phenol or phenol is understood to include solvents with properties similar to phenol such as benzyl alcohol and 2-phenylethanol.

L. Activity of Alfalfa Melanin is Substantially Enhanced by Elicitors

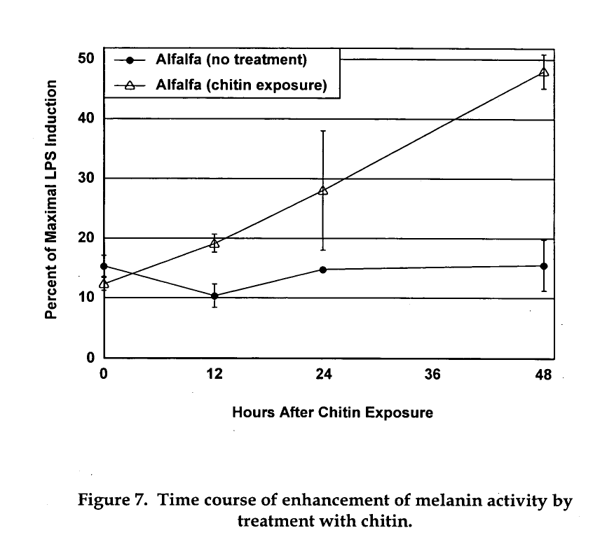

Plant defense strategies against pathogens involves protective mechanisms that are both structural and chemical. Compounds produced by certain soil microbes, cell wall fragments and host-induced endogenous signaling compounds can serve as mediators for enhancing production of secondary metabolites in plants. Secondary metabolites play a pivotal role in plant survival and adaptation, protecting plants against herbivores, pathogen attack and inter-plant competition. They can also serve as growth regulators and as signaling compounds for inducing chemical defense. Although one would not consider melanin to be a secondary metabolite, its production and activity may be controlled by similar signaling mechanisms involved in plant defense. Alfalfa sprouts were used as a test system in this regard since they contained very active melanin and are easy to propagate in vitro. Alfalfa seeds were germinated until approximately 1 inch in length and then treated with known elicitors for 12 hours. The sprouts were grown for an additional 48 hours and melanin was extracted using the phenol procedure. Melanin extracted from alfalfa sprouts that had been treated with chitin exhibited activities in the monocyte assay approximately 10-100 times greater than melanin from untreated sprouts. Other elicitors (salicylic acid or methyl jasmonate) increased the activity of the extracted melanin by 3 to 10 times. FIG. 7 shows the time course of the induction of this heightened melanin activity after treatment with chitin. The EC₅₀ values at the 48 hour time point were 100 ng/ml and 10,000 ng/ml, for chitin and untreated respectively (a doubling of percent activation requires an order of magnitude increase in melanin concentration). Treatment of cultivated botanicals with standard elicitors represents a viable method for enhancing the immunostimulatory activity of melanin within these plants.

Agents that can be used for elicitation in this embodiment and for the plant products used in connection with this invention in general include known elicitors of secondary metabolite production, or systemic acquired resistance products (SAR). More specifically, the elicitors may include, for example, one of the following or any combination of the following: chitin, salicylic acid, methyl jasmonate, glucan, UV light, beta-amino butyric acid, physical damage (wounding) and others known in the art.

M. Oral intake of Melanin Preparations Enhances Immune Parameters in Mice

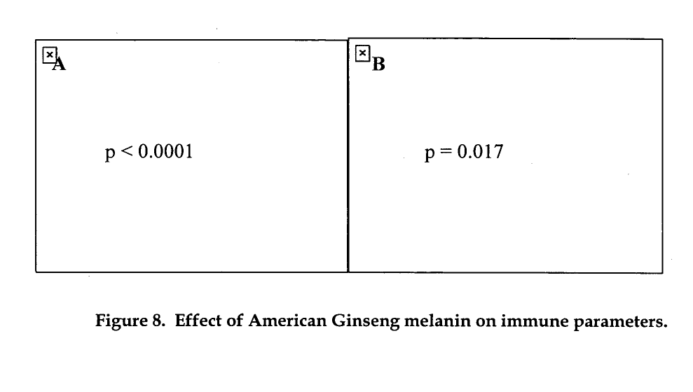

A mouse model was used to demonstrate that oral ingestion of melanin could impact the immune system. One of the parameters examined in this study was IgA production from cells isolated from the Peyer's patches of the small intestine. IgA secreted by the small intestine prevents the adherence of viruses, bacteria and toxic molecules to the mucosal surfaces and is thought to play a major role in eliminating infectious agents. FIG. 8A shows that IgA production from Peyer's patch cells isolated from mice that had consumed melanin extracted from American Ginseng produced more IgA in culture than cells from untreated mice. The inventors also monitored interferon gamma production from spleen cells isolated from these mice. FIG. 8B shows that spleen cells from mice that had consumed American Ginseng melanin produced more interferon gamma in culture than cells from untreated mice.

With respect to the preparations of the present invention, as stated above, an embodiment of the present invention is an immunostimulatory composition that comprises an immunostimulating effective amount of a melanin preparation. Herein, an immunostimulating effective amount is an amount sufficient to activate immune cells, or an amount sufficient to induce immunostimulating activity. Although other immunostimulatory activity is contemplated, in embodiments of the present invention, the immunostimulatory composition of claim 1, wherein the immunostimulation is manifested by monocyte activation.

The melanin preparations of the present invention can be administered to a subject. The term “subject” as used herein specifically includes, for example, human beings, mammals, reptiles, fishes, pets, birds, domesticated animals, farm animals, animals and other living organisms.

The preparation may comprise whole plant material or an extract of a plant. In embodiments of the present invention, the whole plant material or extract may be from one of the following botanicals and any combination thereof: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

A plant material is understood to include, but not limited to, botanicals, dietary supplements, herbs, edible fungi and mushrooms.

The melanin preparation of the present invention is one that yields the following degredation products, when subjected to pyrolysis-GC-MS: toluene; phenol, 4-methylphenol, indole, 7-methylindole. In embodiments of the present invention, the degredation products further comprise: ethylbenzene, 3-methylpyrrole, styrene, benzene acetonitrile, benzene propanenitrile. The melanin preparation does not have to be pure or substantially pure in practice. Embodiments of the present invention include preparation that have a protein content ranging from 0 to about 99%. However, a pure (i.e., protein free) melanin preparation will have the above yields. Additionally, immunostimulatory composition of the present invention may be an aqueous phenol extract.

In addition to the above compositions, another embodiment of the present invention is an immunostimulatory agent comprising a melanin preparation as described herein.

The agents and compositions of the present invention may comprise a carrier or excipient. Further, embodiments of the present invention may be in the form of tablets, dragees, gelules, granules, solutions, syrups, suppositories, lyophilized or non-lyophilized injectable preparations, ovules, creams, pomades, lotions, drops, collyriums, aerosols, and other known delivery mechanisms and may be prepared and administered in the usual manner. Examples of suitable excipients are talc, arabic gum, lactose, starch, magnesium stearate, cacao butter, aqueous or non-aqueous vehicles, fatty bodies of animal or vegetables origin, paraffinic derivatives, glycols, diverse wetting agents, dispersants or emulsifiers and preservatives. The exicipient or delivery system is not know to be critical, and may vary with the only limitation being it must not destroy the immunostimulating activity of the present invention and must have a good tolerance to warm-blooded animals, including humans.

The agents or compositions of the present invention may be, or be part of a pharmaceutical composition, which will also comprise carriers or excipients that facilitate the processing of the present invention. Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. That is, the amount of composition administered will be dependent upon the condition being treated, the subject being treated, on the subject's weight, the severity of the affliction, the manner of administration and the judgment of the individual's physician. The pharmaceutical compositions of the present invention may be manufactured in the manner exemplified in patent application publication No. U.S. 2004/0038931, incorporated herein by reference.

Additionally, the agents and compositions of the present invention are useful as a component of a dietary supplement. Dietary supplements suitable for use in the present invention include compositions wherein the active ingredients are contained in an effective amount to achieve its intended purpose. More specifically, an effective amount means an amount effective to prevent development of or to alleviate the existing symptoms of the subject being treated. Determination of the effective amounts is well within the capability of those skilled in the art, especially in light of the detailed disclosure provided herein. The amount of composition administered will be dependent upon the condition being treated, the subject being treated, on the subject's weight, the severity of the affliction, the manner of administration and the judgment of the individual's physician.

The ingredients of the dietary supplement of this invention are contained in acceptable excipients and/or carriers for oral consumption. The actual form of the carrier, and thus, the dietary supplement itself, may not be critical. The carrier may be a liquid, gel, gelcap, capsule, powder, solid tablet (coated or non-coated), tea or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, agar, lactose, methylcellulose, povidone, carboxymethylcellulose, corn starch, and the like (including mixtures thereof). The various ingredients and the excipient and/or carrier are mixed and formed into the desired form using conventional techniques. Dose levels/unit can be adjusted to provide the recommended levels of ingredients per day in a reasonable number of units.

The dietary supplement may also contain optional ingredients including, for example, herbs, vitamins, minerals, enhancers, colorants, sweeteners, flavorants, inert ingredients, and the like. Such optional ingredients may be either naturally occurring or concentrated forms. Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults.

References

Throughout this disclosure, there are various references to patents and/or printed publications, specifically including the list of references below. All such patents and publications are incorporated herein by reference in their entirety, and are considered as being part of this disclosure.

Adams D O, Hamilton T A (1992). Molecular basis of macrophage activation: diversity and its origins. In The Natural Immune System: The Macrophage; Lewis C E, McGee J O'D, Eds.; Oxford University Press Inc.: New York, NY; pp 75-114.

Baeuerle P A, Henkel T (1994). Function and activation of NF-□B in the immune system. Annu. Rev. Immunol. 12: 141-179.

Bauer R (1999). Chemistry, analysis and immunological investigations of Echinacea phytopharmaceuticals. In Immunomodulatory Agents from Plants; Wagner H, Ed.; Birkhauser Verlag: Basel, Switzerland; pp. 41-88.

Bauer R (2000). Chemistry, pharmacology and clinical applications of Echinacea products. In Herbs, Botanicals & Teas; Mazza G, Oomah B D, Eds.; Technomic Publishing Company Inc.: Lancaster, Pa.; pp. 45-73.

Bauer R, Jurcic K, Puhlmann J, Wagner H (1988a). Immunologische In-vivo- und In-vitro-Untersuchungen mit Echinacea-Extrakten. Arzneim-Forsch. 38: 276-281.

Bauer R, Khan I A, Wagner H (1988b). TLC and HPLC analysis of Echinacea pallida and E. angustifolia roots. Planta Med. 54, 426-430.

Bauer R, Remiger P (1989). Der Einsatz der HPLC bei der Standardisierung von Echinacea-Drogen. Arch. Pharm. 322: 324.

Bauer R, Remiger P, Jurcic K, Wagner H (1989a). Beeinflussung der Phagozytose-Aktivitat durch Echinacea-Extrakte. Z. Phytother. 10: 43-48.

Bauer R, Remiger P, Wagner H (1988c). Echinacea. Comparative TLC and HPLC analysis of herbal drugs from Echinacea purpurea, E. pallida and E. angustifolia. Part 3. Dtsch. Apoth. Ztg. 128: 174-180.

Bauer R, Remiger P, Wagner H (1989b). Alkamides from the roots of Echinacea angustifolia. Phytochemistry 28: 505-508.

Clifford L J, Nair M G, Rana J, Dewitt D L (2002). Bioactivity of alkamides isolated from Echinacea purpurea (L.) Moench. Phytomedicine 9: 249-253.

Copeland R A (1994). Peptide mapping and amino acid analysis. In Methods for Protein Analysis: A Practical Guide to Laboratory Protocols; Chapman & Hall: New York, N.Y., Chapter 7, pp. 126-150.

Dzierzega-Lecznar A, Chodurek E, Stephen K, Wilczok T (2002). Pyrolysis-gas-chromatography-mass spectrometry of synthetic neuromelanins. J. Anal. Appl. Pyrol. 62:239-248.

Emmendorffer A C, Wagner H, Lohmann-Matthes M-L (1999). Immunologically active polysaccharides from Echinacea purpurea plant and cell cultures. In Immunomodulatory Agents from Plants; Wagner H, Ed.; Birkhauser Verlag: Basel, Switzerland; pp. 89-104.

Galanos C, Luderitz O, Westphal 0 (1969). A new method for the extraction of R lipopolysaccharides. European J Biochem. 9: 245-249.

Goel V, Chang C, Slama J V, Barton R, Bauer R, Gahler R, Basu T K (2002). Alkylamides of Echinacea purpurea stimulate alveolar macrophage function in normal rats. Int. Immunopharmacol. 2: 381-387.

Gomez B L, Nosanchuk J D (2003). Melanin and fungi. Curr. Opin. Infect. Dis. 16: 91-96.

Housby J N, Cahill C M, Chu B, Prevelige R, Bickford K, Stevenson M A, Calderwood S K (1999). Non-steroidal anti-inflammatory drugs inhibit the expression of cytokines and induce HSP70 in human monocytes. Cytokine 11: 347-358.

Hung Y -C, Sava V M, Juang C -L, Yeh T -C, Shen W -C, Huang G S (2002). Gastrointestinal enhancement of MRI with melanin derived from tea leaves (Thea sinesis Linn.). J Ethnopharmacol. 79: 75-79.

Kamei H, Koide T, Kojima T, Hasegawa M, Umeda T (1997). Suppression of growth of cultured malignant cells by allomelanins, plant-produced melanins. Cancer Biother. & Radiopharm. 12: 47-49.

Knicker H, Almendros G, Gonzalez-Vila F J, Ludemann H D, Martin F (1995). ¹³C and ¹⁵N NMR analysis of some fungal melanins in comparison with soil organic matter. Org. Goechem. 23: 1023-1028.

Krysciak J (1985). Solubility of melanin and humin preparations in polar aromatic solvents. Folia Biol. 33: 195-214.

Lohmann-Matthes M -L, Wagner H (1989). Aktivierung von Makrophagen durch Polysaccharide aus Gewebekulturen von Echinacea purpurea. Z. Phytother. 10: 52-59.

Luettig B, Steinmuller C, Gifford G E, Wagner H, Lohmann-Matthes M -L (1989). Macrophage activation by the polysaccharide arabinogalactan isolated from plant cell cultures of Echinacea purpurea. J. Natl. Cancer Inst. 81: 669-675.

May M J, Ghosh S (1998). Signal transduction through NF-kappa B. Immunol. Today, 19: 80-88.

Melchart D, Clemm C, Weber B, Draczynski T, Worku F, Linde K, Weidenhammer W, Wagner H, Saller R (2002). Polysaccharides isolated from Echinacea purpurea herba cell cultures to counteract undesired effects of chemotherapy—a pilot study. Phytother. Res. 16: 138-42.

Montefiori D C, Zhou J Y (1991). Selective antiviral activity of synthetic soluble L-tyrosine and L-dopa melanins against human immunodeficiency virus in vitro. Antiviral Res. 15: 11-25.

Muller-Jakic B, Breu W, Probstle A, Redl K, Greger H, Bauer R (1994). In vitro inhibition of cyclooxygenase and 5-lipoxygenase by alkamides from Echinacea and Achillea species. Planta Med. 60: 37-40.

Pietta P, Mauri P, Bauer R (1998). MEKC Analysis of different Echinacea species. Planta Med. 64: 649-652.

Proksch A, Wagner H (1987). Structural analysis of a 4-O-methylglucuronoarabinoxylan with immunostimulating activity from Echinacea purpurea. Phytochemistry 26: 1989-1993.

Pugh N, Ross S A, ElSohly H N, ElSohly M A, Pasco D S (2001). Isolation of three high molecular weight polysaccharide preparations with potent immunostimulatory activity from Spirulina platensis, Aphanizomenon flos-aquae and Chlorella pyrenoidosa. Planta Med. 67: 737-742.

Riley P A (1997). Melanin. Int. J Biochem. Cell Biol., 29 (11): 1235-1239.

Roesler J, Steimnuller C, Kiderlen A, Emmendorffer A, Wagner H, Lohmann-Matthes M -L (1991). Application of purified polysaccharides from cell cultures of the plant Echinacea purpurea to mice mediates protection against systemic infections with Listeria monocytogenes and Candida albicans. Int. J. Immunopharmacol. 13: 27-37.

Sava, V. M., Galkin, B. N., Hong, M -Y, Yang, P -C, Huang, G. S. (2001) A novel melanin-like pigment derived from black tea leaves with immuno-stimulating activity. Food Research International 34: 337-343,

Steinmuller C, Roesler J, Grottrup E, Franke G, Wagner H, Lohmann-Matthes M -L (1993). Polysaccharides isolated from plant cell cultures of Echinacea purpurea enhance the resistance of immunosuppressed mice against systemic infections with Candida albicans and Listeria monocytogenes. Int. J Immunopharmacol. 15: 605-614.

Stimpel M, Proksch A, Wagner H, Lohmann-Matthes M- L (1984). Macrophage activation and induction of macrophage cytotoxicity by purified polysaccharide fractions from the plant Echinacea purpurea. Infect. Immun. 46: 845-849.

Vas G, Vekey K, Czira G, Tamas J, Favretto D, Traldi P, Bertazzo A, Costa C, Allegri G (1993). Characterization of melanins by pyrolysis/gas chromatography/mass spectrometry. Rapid. Commun. Mass Spectrom., 7: 870-873.

Wagner H, Proksch A (1981). Uber ein immunstimulierendes Wirkprinzip aus Echinacea purpurea MOENCH. Z. Angew. Phytother. 2: 166-171.

Wagner H, Proksch A, Riess-Maurer I, Vollmar A, Odenthal S, Stuppner H, Jurcic K, LeTurdu M, Fang J N (1985). Immunstimulierend wirkende Polysaccharide (Heteroglykane) aus hoheren Pflanzen. Arzneim.-Forsch. 35: 1069-1075.

Wagner H, Stuppner H, Schafer W, Zenk M A (1988). Immunologically active polysaccharides of Echinacea purpurea cell cultures. Phytochemistry 27: 119-126.

Westphal O, Jann K (1965). Bacterial lipopolysaccharides: Extraction with phenol-water and further applications of the procedure. In Methods in Carbohydrate Chemistry: Volume V, General Polysaccharides; Whistler R L, Ed.; Academic Press: New York, N.Y.; pp 83-91.

Zecca L, Mecacci C, Seraglia R, Parati E (1992). The chemical characterization of melanin contained in substantia nigra of human brain. Biochim. Biophys. Acta, 1138: 6-10.

The invention thus being described, it would be obvious that the same may be varied in many ways without departing from the scope of the present invention. All such variations as would be obvious to one of ordinary skill in the art are considered as being part of the present invention.

Unless otherwise indicated, all numbers expressing quantities of ingredients, properties such as reaction conditions, and so forth used herein are to be understood as being modified in all instances by the term “about.” Accordingly, unless indicated to the contrary, the numerical parameters set forth herein are approximations that may vary depending upon the desired properties sought to be determined by the present invention.

Notwithstanding that the numerical ranges and parameters setting forth the broad scope of the invention are approximations (particularly in the disclosure, above), the numerical values set forth in the disclosure/experimental or example sections are reported as precisely as possible. Any numerical value, however, inherently contain certain errors necessarily resulting from the standard deviation found in their respective testing measurements.

1. An immunostimulatory composition that comprises an immunostimulating effective amount of a melanin preparation as an extract of one of the following botanicals and any combination thereof: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

2. The immunostimulatory composition of claim 1, wherein the melanin preparation comprises an extract of Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola.

3. The immunostimulatory composition of claim 1, wherein the melanin preparation yields at least one of following degradation products, when subjected to pyrolysis-GC-MS: toluene, phenol, 4-methylphenol, indole, 7-methylindole, ethylbenzene, 3-methylpyrrole, styrene, benzene acetonitrile, benzene propanenitrile.

4. The immunostimulatory composition of claim 3, wherein the melanin preparation is protein-free.

5. The immunostimulatory composition of claim 1, wherein said melanin preparation results from an aqueous phenol extract or phenol extract.

6. The immunostimulatory composition of claim 1, wherein the melanin preparation results from an extract produced by extraction with water, alcohol, weak base or any combination thereof.

7. The immunostimulatory composition of claim 1, further comprising a carrier or excipient.

8. The immunostimulatory composition of claim 1, wherein the immunostimulation is manifested by monocyte activation.

9. A method of treating a subject requiring immune mediation comprising administering to said subject an immunostimulatory cell activating amount of a melanin preparation of claim 1.

10. An immunostimulatory agent, comprising:
an immunostimulatory effective amount of a melanin preparation, wherein
the melanin preparation comprises an extract from at least one of the following botanicals: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom), Boletus edulis (porcini mushroom).

11. The immunostimulatory agent of claim 10, wherein the melanin preparation comprises an extract of Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola.

12. The immunostimulatory agent of claim 10, wherein the melanin preparation yields at least one of the following degradation products, when subjected to pyrolysis-GC-MS: toluene, phenol, 4-methylphenol, indole, 7-methylindole, ethylbenzene, 3-methylpyrrole, styrene, benzene acetonitrile, benzene propanenitrile.

13. The immunostimulatory agent of claim 12, wherein the melanin preparation is protein-free.

14. The immunostimulatory agent of claim 10, wherein the melanin preparation results from an aqueous phenol extract or phenol extract.

15. The immunostimulatory agent of claim 10, wherein the melanin preparation results from an extract produced by extraction with water, alcohol, weak base or any combination thereof.

16. A method of activating an immune cell having a receptor that recognizes melanin in a subject, comprising:
providing an effective immune cell activating amount of the composition of claim 1; and
introducing said extract to said subject.

17. The method of claim 16, wherein the immune cell is a monocyte.

18. A method of measuring an immunostimulating effective amount of an immunostimulating melanin, comprising the following steps:
a. providing a plant material selected from the group of Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom), Boletus edulis (porcini mushroom);
b. extracting the plant material with a solvent;
c. collecting precipitates comprising melanin;
d. removing contaminants by a solvent wash;
e. removing contaminants by solvent partitioning;
f. collecting precipitates comprising melanin;
g. optionally testing the collected precipitates for activation of immune cells;
h. optionally assigning an immunostimulatory value to said melanin preparation to be used as a standard.

19. The method of claim 18, wherein the solvent in step (b) is at least one of phenol, aqueous phenol, alcohol, water, weak base, or any combination thereof.

20. The method of claim 18, wherein in step (e) the partitioning is phenol:water partitioning.

21. The method of claim 18, wherein step (e) comprises phenol/chloroform: water partitioning.

22. The method of claim 18, wherein step (e) removes protein contaminants to provide a protein-free melanin precipitate.

23. The method of claim 22, further comprising an amino acid test analysis step to confirm protein content.

24. A method for employing a standard for measuring an immunostimulating effective amount of a melanin preparation, comprising:
a. providing a standard melanin preparation with an immunostimulatory value;
b. providing a plant material;
c. extracting the plant material with a solvent;
d. collecting precipitates comprising melanin;
e. removing contaminants by a solvent wash;
f. removing contaminants by solvent partitioning;
g. collecting precipitates comprising melanin;
h. testing the collected precipitates for activation of immune cells;
i. comparing the activity of the collected precipitates to the standard melanin preparation immunostimulatory value.

25. The method of claim 24, wherein the plant material is selected from the group of Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom), Boletus edulis (porcini mushroom), and any combination thereof.

26. The method of claim 24, wherein in step (c) the solvents are at least one of aqueous phenol, phenol, alcohol, water, weak base, or any combination thereof.

27. The method of claim 24, wherein in step (f) the partitioning is phenol:water partitioning.

28. The method of claim 24, wherein step (f) comprises phenol/chloroform: water partitioning.

29. The method of claim 24, wherein step (f) removes protein contaminants to provide a protein-free melanin precipitate.

30. A method for preparing a product containing a standardized amount of immunostimulatory melanin, comprising:
a. providing a standard melanin preparation with an immunostimulatory value;
b. providing a melanin product;
c. extracting the melanin product with a solvent;
d. collecting precipitates comprising melanin;
e. removing contaminants by a solvent wash;
f. removing contaminants by solvent partitioning;
g. collecting precipitates comprising melanin;
h. testing the collected precipitates for activation of immune cells;
i. comparing the activity of the collected precipitates to the standard melanin preparation immunostimulatory value to determine a standardized activity value of the product.

31. The method of claim 30, wherein step (c) the solvents are at least one of aqueous phenol, phenol, alcohol, water, weak base or any combination thereof.

32. The method of claim 30, wherein the melanin product is whole plant material.

33. The method of claim 32, wherein the whole plant material is plant material selected from a plant from at least one of: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom), Boletus edulis (porcini mushroom).

34. The method of claim 33, wherein the melanin product is a melanin extract preparation.

35. The method of claim 34, wherein the melanin extract preparation is an extract of one of the following botanicals and any combination thereof: Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom).

36. A method of treating, preventing, or ameliorating a condition or disease in a subject requiring enhanced immune system support; comprising providing an effective immune cell activating amount of the composition of claim 1, and introducing said composition to said subject.

37. The method of claim 36, wherein the condition or disease is an immune deficiency.

38. The method of claim 36, wherein the condition or disease is cancer.

39. The method of claim 36, wherein the condition or disease is a flngal, viral, or bacterial infection.

40. The method of claim 36, wherein the condition or disease is a wound.

41. A method of preparing an extract enriched for immunostimulatory melanin, comprising:
a. extraction of plant material with a solvent;
b. enriching for immunostimulatory melanin by precipitation and collection of melanin;
c. optionally washing precipitated melanin with a solvent to remove contaminants.

42. The method of claim 41, wherein step (a) comprises a water solvent, alcohol solvent, or a mixture thereof.

43. The method of claim 41, wherein the solvent in step (a) comprises a weak base.

44. The method of claim 41, wherein the solvent in step (a) comprises at least one of a weak base, an alcohol, and mixtures thereof.

45. The method of claim 41, wherein the solvent in step (a) comprises ammonium hydroxide.

46. The method of claim 41, wherein step (b) comprises precipitation of melanin preparation by addition of acid.

47. The method of claim 41, wherein the solvent in step (c) comprises alcohol.

48. A melanin preparation obtained by extracting a plant or edible fungi with a solvent; wherein the melanin preparation is immunostimulatory in vitro and in vivo, and upon pyrolysis-GC-MS yields at least one of the following: toluene, phenol, 4-methylphenol, indole, 7-methylindole, ethylbenzene, 3-methylpyrrole, styrene, benzene acetonitrile, benzene propanenitrile.

49. A method for enhancing the activity of a melanin within a plant, comprising:
(1) providing a plant;
(2) treating said plant with an elicitor of secondary metabolite production.

50. The method of claim 49, further comprising extracting melanin from elicitor treated plant.

51. The method of claim 49, wherein the elicitor is chitin.

52. The method of claim 49, wherein the elicitor comprises at least one the following: chitin, salicylic acid, methyl jasmonate, glucan, UV light, beta-amino butyric acid, or physical damage (wounding).
